(19) United States
(12) Patent Application Publication
(10) Pub. No.: US 2006/0200029 A1
(43) Pub. Date: Sep. 7, 2006

(54) Universal transportable vital signs monitor

(75) Inventors: Kevin Ray Evans, New Richmond, WI (US); Matthew J. Hill, Saint Paul, MN (US); Robert S. Bryngelson, Saint Paul, MN (US); Victor F. Glava, Little Canada, MN (US)

Correspondence Address: ALTERA LAW GROUP, LLC, 6500 CITY WEST PARKWAY, SUITE 100, MINNEAPOLIS, MN, 55344-7704, US

(73) Assignee: Medwave, Inc.

(21) Appl. No.: 11/138,953

(22) Filed: May 26, 2005

Related U.S. Application Data
(63) Continuation-in-part of application No. 11/072,199, filed on Mar. 4, 2005.

(51) Int. Cl. A61B 5/02

(52) U.S. Cl. 600/485

(57) ABSTRACT

A transportable vital signs monitor accommodates patients over a broad range of body sizes. The monitor has various universal vital signs sensor units attached to it, such as sensor units for blood oxygen saturation, temperature, and non-invasive blood pressure. The monitor has a graphical display and may have alphanumeric displays. The graphical display is for visually displaying various waveforms and other information of use to the caregiver such as an SpO2 waveform and a blood pressure waveform trend display. The graphical display may also display alphanumeric information. The transportable vital signs monitor may also include communications capability for transferring the vital signs, and in particular may include a short range capability such as Bluetooth for peer-to-peer communication of vital signs.

### CROSS-REFERENCE TO RELATED APPLICATIONS

This application is a continuation-in-part of U.S. patent application Ser. No. 11/072,199 filed Mar. 4, 2005, which names Kevin R. Evans as inventor and is entitled “Articulated placement guide for sensor-based noninvasive blood pressure monitor,” which hereby is incorporated herein in its entirety by reference thereto.

### BACKGROUND OF THE INVENTION

1. Field of the Invention

The present invention relates to patient vital signs monitors, and more particularly to vital signs monitors that operate over a broad range of patient sizes.

2. Description of the Related Art

In many situations, particularly emergency situations such as ambulance transport and the emergency room, the monitoring of a patients vital signs, such as temperature, oxygen saturation, and blood pressure, is important. For proper care, it is important to monitor these vital signs over a period of time, so that any appropriate actions may be taken in response to trends in the vital signs.

A patient's body core temperature is typically measured via the inner ear, which responds to changes in core temperature more quickly than most other body parts. A probe is inserted into the ear and placed in contact or in close proximity to the tympanic membrane of the ear. The tympanic membrane acts a radiator of blackbody radiation of a particular temperature, with a characteristic spectrum that depends on the radiator. Electrical signals are delivered from the probe via one or more wires to a processor, typically located away from the probe (as opposed to located in close proximity to the ear). The processor converts the signals from the probe into a temperature value that may be read visually by the staff of the hospital. Additionally, the temperature values over a period of time may be stored or displayed by the processor, so that trends may be detected.

Oxygen saturation, known equivalently as SpO₂, is commonly measured by a probe that clips onto the fingertip of a patient. The probe typically has a pair of light-emitting diodes with two different wavelengths, usually one in the red and the other in the near-infrared. The diodes illuminate a patch of skin, and the probe has one or more photodetectors or wavelength-sensitive filters that detect the amount of light reflected at each wavelength. The spectrum of the reflected light depends on the amount of oxygen contained in the blood, in the same manner that oxygen-rich blood visually appears bright red, while oxygen-depleted blood appears a much darker shade of red. By comparing the relative reflections at each wavelength, the amount of oxygen may be determined. The probe is usually connected by one or more wires to a processor, which can also store or display the SpO₂ values over a period of time.

Blood pressure is commonly measured noninvasively by the use of an oscillatory cuff. A cuff operates in accordance with either an oscillometric or ausculatory method. However, since the oscillometric and auscultatory methods require inflation of the cuff, these methods are not entirely suitable for performing frequent measurements and measurements over long periods of time. The frequency of measurement is limited by the time required to inflate and deflate the cuff, and the pressure imposed by the cuff is uncomfortable to the patient and occludes the artery, thereby affecting any “downstream” measurements such as oxygen saturation. Moreover, both the oscillometric and auscultatory methods lack accuracy and consistency. Another disadvantage of the cuff is that it must be made available in numerous sizes to accommodate different patients. Commonly cuffs are provided in six different sizes. Typically all of the different cuffs must be readily available to the practitioner, resulting in unnecessary effort for the practitioner. If the different cuff sizes are stored with the instrument, this unnecessarily increases the size of the storage case.

The cuff is also quite disadvantageous when used on morbidly obese patients. Regardless of how a cuff is sized for the patient, the cuff yields inaccurate results and tends to injure the soft tissues of the patient.

While blood pressure may be measured noninvasively using a cuff, a superior approach for the noninvasive monitoring of blood pressure applies a pressure sensor to the patient's wrist over the radial artery with a varying hold-down force, so that the sensor presses the artery against the radius bone. The sensor should be positioned at the distal edge of the radius bone. Devices of this type and their associated methods of calculating blood pressure are described in various patents, including the sensor described in U.S. Pat. No. 5,450,852 entitled “Continuous Non-Invasive Blood Pressure Monitoring System” which issued Sep. 19, 1995 to Archibald et al.; the basic algorithm described in U.S. Pat. No. 5,797,850 issued Aug. 25, 1998 to Archibald et al., the beat onset detection method as described in U.S. Pat. No. 5,720,292 issued Feb. 24, 1998 to Poliac, and the segmentation estimation method as described in U.S. Pat. No. 5,738,103 issued Apr. 14, 1998 to Poliac, all of which are incorporated herein in their entirety by reference thereto. Commercially available devices of the sensor-based type include the Vasotrac®) model AMP205A NIBP monitor system, which is available from Medwave Inc. of Danvers, Mass. Revision K of the Vasotrac monitor uses a manual motion compensation technique, while Revision L uses an automatic motion compensation technique.

The sensor-based type of device is advantageous over the cuff in many respects, being both accurate with a typical mean correlation of about 0.97 with a well managed arterial line, as well as being fast with the ability to calculate four accurate readings of systolic, diastolic, and mean pressure and heart rate per minute. Moreover, some versions of the device are able to store and display full pulse arterial waveforms. The sensor-based type of device is also convenient for the patient. Because the device uses a relatively small soft-surfaced sensor placed over the radial artery at the wrist, the patient does not experience the discomfort of a fully occluded artery and need not remove any clothing or roll his/her sleeve to the upper arm. Unlike other techniques such as the cuff, operation with the sensor-type device is smooth with little noise, so it generally does not disturb patients who are resting.

The sensor-based type of device has also been found to provide significantly more accurate values compared to the upper arm oscillometric cuff pressure monitoring. While pressure monitoring using the arterial canula is still the gold standard of blood pressure measurement, the sensor-based type of device should be a valuable tool for monitoring the blood pressure of morbidly obese patients perioperatively without the possible negative side effects of the arterial canula.

In order to ensure an accurate reading, the sensor should be placed accurately and stabilized at the distal edge of the radius bone using a placement guide. The placement guide for the Vasotrac monitor is provided in different sizes, corresponding to the circumference of the patient's wrist. An “adult normal” size corresponds to wrist circumferences of about 15 to 18 cm, a “large adult” size corresponds to wrist circumferences of about 18 to 22 cm, and a “pediatric” size corresponds to wrist circumferences of about 11 to 15 cm, for example. These three sizes cover most or all of the normal ranges of wrist sizes of patients. However, somewhat disadvantageously, the need for different placement guides to accommodate the various ranges in the sizes of patients' arms requires that three different parts are be manufactured, stocked in inventory, and provided with the monitoring device.

While temperature, oxygen saturation, and blood pressure measuring devices are available as separate systems, they have been integrated into single systems generally known as vital signs monitors, and have also been integrated along with other measurements such as ECG into single systems known as bedside monitors. Such monitors are available from various manufactures, including Welch Allyn Inc. of Beaverton, Oreg., and Nihon Kohden America, Inc. of Foothill Ranch, Calif. The Vital Signs Monitor 300 Series available from Welch Allyn, for example, is configurable for noninvasively measuring blood pressure with a cuff, as well as pulse oximetry and temperature. No waveforms are displayed. The Vital Signs Monitor Model OPV1500 available from Nihon Kohden America, for example, noninvasively measures blood pressure with a cuff, and may also perform pulse oximetry and ECG measurements. The information displayed is a respiration number and an ECG waveform, an SpO₂ number and an SpO₂ waveform, and pulse rate, systolic pressure, diastolic pressure, and mean pressure numbers. An example of a full featured bedside monitor is the Procyon series monitor, available from Nihon Kohden America. The Procyon monitor can simultaneously accept the inputs from various devices designed to measure ECG/respiration, non-invasive blood pressure), BP, ETCO₂, FiO₂, temperature, and cardiac output. The configurable screen can display a plethora of information. However, inasmuch as cuffs do not provide pulse waveform information, none of these monitors can display pulse waveform information (as opposed to the heart's electrical activity as reported by an ECG) from which the mechanical activity of the patient's heart can be observed.

While some of the previously-discussed monitors are portable in that they can be easily moved from bedside to bedside, other bedside monitors are much larger in size and weight and so typically are meant to be left in place for some time. An example of such a monitor is the Model BSM-400 bedside monitor, which is available from Nihon Kohden Corporation of Tokyo, Japan. The model BSM-400 bedside monitor performs a great many different measurements, including the noninvasive measurement of blood pressure with a cuff. The monitor also features a modular design which accommodates a sensor-based noninvasive blood pressure monitor module such as the model MJ23 CNIBP OEM Module, which is available from Medwave Inc. of Danvers, Mass. While the model BSM-400 as equipped with the model MJ23 CNIBP OEM module is able to display pulse waveform information, such a monitor is not well suited for environments in which portability is needed, and is not at all suitable for transport monitoring.

Transport monitoring and emergency room monitoring provide challenges in addition to those normally faced by bedside monitors. Not only is the instrumentation used to measure vital signs during transport and emergency situations subject to additional stresses, but the caregivers involved in transport and emergency monitoring have precious little time to customize the instrumentation to the size of the patient, or to look for different size pieces of the instrumentation that may have been misplaced or lost. The pressure cuff is an apt example of a part of the instrumentation that must be provided in a number of different sizes, which creates clutter about the instrument, costs the caregiver time to select and assemble, and creates the possibility that the right size will not be available when needed due to the part having been misplaced or omitted from the kit.

Another challenge imposed by transport monitoring is related to ownership of equipment. If a patient is discovered at home and brought to the hospital in an ambulance, for example, the vital signs monitor and connected measuring devices typically are owned by the ambulance company and remain with the ambulance. When the patient is transferred from the ambulance to the emergency room, the measuring devices are removed from the patient, and new measuring devices attached to the hospital's vital signs monitor are applied to the patient. Information acquired during the ambulance trip either is lost, or is printed out by the ambulance crew and furnished in paper form to the hospital. Furthermore, individual departments inside the hospital may own their own monitors, and a handoff between departments may occur several times during the treatment of the patient, requiring a change-out of the measuring devices. In such a change out, prior vital signs information is not lost if the vital signs monitors are networked to the internal hospital network, but not all hospitals and clinics can afford this capability.

### BRIEF SUMMARY OF THE INVENTION

What is needed is an improved vital signs monitor suitable for transport and emergency monitoring as well as bedside monitoring. It would be advantageous for such a vital signs monitor to have universal measuring devices, including measuring devices that are not harmful to morbidly obese patients, so that only a single measuring device of each type suitable over a substantial range of patient sizes need be provided in the system. Each of the measuring devices should be “hardened” against high motion conditions so that vital signs may be acquired during transport in high motion environments such as by ambulance and aircraft, or by stretcher or gurney over rough or uneven ground. Moreover, it would be advantageous for such a vital signs monitor to be able to acquire vital signs data previously acquired by other vital signs monitors over the course of monitoring an event, so that a continuous or nearly continuous history of the patient's vital signs over the duration of the event is available to the caregiver from the last vital signs monitor in the sequence.

These and other advantages are realized individually or collectively in varying degrees by the various embodiments of the present invention. One embodiment of the present invention is an apparatus for monitoring vital signs of pediatric, adult, and morbidly obese patients, comprising a transportable control unit; a universal noninvasive patient blood oxygen saturation sensor unit coupled to the control unit; a universal noninvasive patient temperature sensor unit coupled to the housing; and a universal patient noninvasive blood pressure sensor unit coupled to the housing.

Another embodiment of the present invention is an apparatus for monitoring vital signs of pediatric, adult, and morbidly obese patients, comprising a transportable control unit having a display; a noninvasive patient blood pressure sensor unit usable without modification over a range of pediatric, adult and morbidly obese patients, the control unit being suitable for wrist sizes over a range of about 11 cm to about 22 cm and coupled to the control unit; a patient blood oxygen saturation sensor unit usable without modification over the range of pediatric, adult and morbidly obese patients and coupled to the control unit; and a patient temperature sensor unit usable without modification over the range of pediatric, adult and morbidly obese patients and coupled to the control unit. The control unit comprises processing circuitry for generating motion-compensated pressure waveform trend data from measurements by the blood pressure sensor unit, for calculating motion-compensated systolic pressure, diastolic pressure, and mean pressure values from measurements by the blood pressure sensor unit, for generating pulse oximetry waveform data from measurements by the blood oxygen saturation sensor unit, and for calculating oxygen saturation values from measurements by the blood oxygen saturation unit. The control unit further comprises display circuitry for displaying the pressure waveform trend data, the pulse oximetry waveform data, and the oxygen saturation, systolic pressure, diastolic pressure, and mean pressure values on the display.

Another embodiment of the present invention is an apparatus for monitoring vital signs of a patient, comprising a transportable housing; a clip-type fingertip patient blood oxygen saturation sensor mechanically coupled to the housing; a tympanic patient temperature sensor mechanically coupled to the housing; a noninvasive wrist-mounted blood pressure sensor unit mechanically coupled to the housing, the blood pressure sensor comprising an articulated placement guide suitable for wrist sizes over a range of about 11 cm to about 22 cm; a display mounted in the housing, the display having an LCD portion for displaying information graphically, and an LED portion for displaying information alphanumerically; processing circuitry contained in the housing and electrically coupled to the blood oxygen saturation sensor, the temperature sensor, and the noninvasive blood pressure sensor for determining patient blood oxygen saturation, patient temperature, and patient blood pressure; display circuitry contained in the housing and electrically coupled to the processing circuitry for displaying waveforms indicative of the patient blood oxygen saturation and the patient blood pressure on the LCD portion of the display, and for displaying alphanumeric values indicative of the patient blood oxygen saturation, patient temperature, and the patient blood pressure on the LED portion of the display; and communications circuitry contained in the housing and electrically coupled to the processing circuitry for transmitting the patient blood oxygen saturation, the patient temperature, and the patient blood pressure using a wireless protocol.

Another embodiment of the present invention is a method of monitoring a patient for vital signs, comprising acquiring vital signs of a patient during transport in a motorized vehicle with a transportable vital signs monitor system having universal vital sign sensors; and continuing to acquire the vital signs of the patient with the transportable vital signs monitor system and the universal vital sign sensors in an emergency room.

Another embodiment of the present invention is a method of monitoring a patient for vital signs, comprising acquiring a first set of vital signs of a patient in a motorized vehicle with a first transportable vital signs monitor system having universal vital sign sensors; acquiring a second set of vital signs of the patient with a second transportable vital signs monitor system having universal vital sign sensors in a stationary examination location; and transferring the first set of vital signs from the first transportable vital signs monitor to the second transportable vital signs monitor while the patient is being transferred from the motorized vehicle to the stationary examination location.

### BRIEF DESCRIPTION OF THE SEVERAL VIEWS OF THE DRAWINGS

FIG. 1 is a schematic drawing of a transportable universal vital signs monitor.

FIG. 2 is a schematic diagram of an application for the vital signs monitor of FIG. 1.

FIG. 3 is a schematic diagram of another application for the vital signs monitor of FIG. 1.

FIG. 4 is a schematic diagram of another application for the vital signs monitor of FIG. 1.

FIG. 5 is a side view of a blood pressure sensor unit with an articulated placement guide that is suitable for the vital signs monitor of FIG. 1.

FIG. 6 is a perspective drawing of the articulated placement guide for the blood pressure sensor unit shown in FIG. 5.

FIG. 7 is a side view drawing of the articulated placement guide for the blood pressure sensor unit shown in FIG. 5.

FIG. 8 is a top view drawing of the articulated placement guide for the blood pressure sensor unit shown in FIG. 5.

### DETAILED DESCRIPTION OF THE INVENTION INCLUDING THE BEST MODE

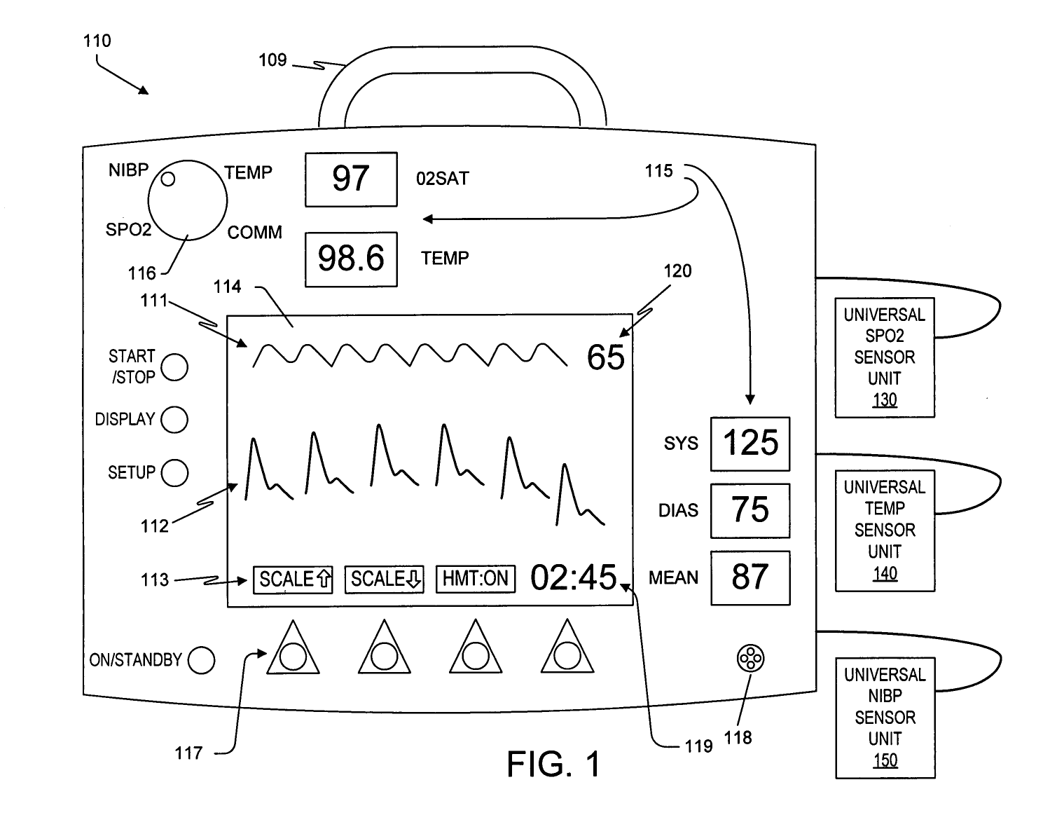

FIG. 1 shows a transportable vital signs monitor 110 that can accommodate patients over a broad range of body sizes. The monitor 110 has a plurality of connectors (not shown) which are configured to accept connections to various universal sensors, preferably vital sign sensors such as blood oxygen saturation (SpO₂) sensor unit 130, temperature sensor unit 140, and non-invasive blood pressure (“NIBP”) sensor unit 150. The monitor 110 also has a graphical display 114, illustratively a LCD display of about 4.5 inches by 2.5 inches, that visually displays various waveforms and other information of use to the caregiver. Shown are an SpO₂ waveform 111 and a waveform trend display 112, which shows the patient's arterial waveform in mmHg and is designed for routine monitoring. The graphical display may also display other information as desired, including programmable labels 113 such as “Scale Up,” “Scale Down,” and “HMT:OFF,” which are respectively associated with keys 117 and which may change with the various display modes of the monitor 110. The keys 117 may be referred to as “soft keys” because of their programmable and changeable functionality. The graphical display may also display alphanumeric information, such as the elapsed time 119 of the current measurement period and patient pulse rate 120. Illustratively, various alphanumeric displays 115 are included for displaying alphanumeric information such as the oxygen saturation value O2SAT, systolic arterial pressure SYS, diastolic arterial pressure DIAS, mean arterial pressure MEAN, and body temperature TEMP. Illustratively, the alphanumeric displays 115 may include light emitting diodes (“LED”). It will be appreciated that the alphanumeric displays 115 may be eliminated and all information furnished on the graphical display 114, or the information display on the graphical display 114 and on the alphanumeric displays 115 may be varied as desired.

During normal operation, the monitor 110 displays the SpO₂ waveform 111, the elapsed time 119, the pulse rate 120, and the alphanumeric displays 115 essentially in real time. The waveform trend display 112 may not be in real time where, as in the technique used in the Vasotrac monitor, each waveform is constructed over a period of about 15 seconds based on multiple sensed pressure waveforms over that period. However, the monitor 110 may be operated in a Real Time Display Mode where the pressure signal as produced by the sweeping action of the sensor unit is displayed. While this mode does show usable arterial waveform information, the scale is not the patient's blood pressure in mmHg. However, the mode may be correlated with the SpO₂ waveform 111.

The monitor 110 has a number of controls 117 that can function differently depending on the mode of operation, which allows a caregiver or patient to change the quantities displayed, the order in which they are displayed, and optionally the time interval that is displayed. Illustratively, the controls also include a rotary dial 116 and various hard keys for start/stop, display, setup, and on/standby. The rotary dial 116 illustratively is used to switch among setup screens for blood oxygen saturation SPO2, temperature TEMP, non-invasive blood pressure NIBP, and communications COMM.

In addition to many of the same screens as are available on the Vasotrac monitor, the monitor 110 has three additional screens displayed on the display 114, one for temperature and two for pulse oximetry. The monitor 110 also has a “Graphical Trend Screen” which displays the previous readings in the form of a linear graph, similar to the Graphical Trend Screen of the Vasotrac monitor. The time period for the graph is selected by the user. There is also a “Trend Table Screen” for each additional feature, showing the exact numeric value and the time and date each measurement was taken in tabular form. Again, this is very similar to the Trend Table Screen available on the Vasotrac monitor. Users can scroll up and down this table to examine the previous readings.

The monitor preferably is small enough, illustratively being on the order of about 12 inches by 12 inches by 12 inches or less, so that it may be placed on nearly any flat surface such as a tabletop. Because the monitor 110 is transportable, illustratively weighing about 11.5 pounds or less, an optional handle 109 is shown extending from the monitor 110. The monitor 110 is also provided with a universal mount (not shown), many suitable types being well known in the art, so that it may be mounted on a pole clamp or on a rail of a gurney.

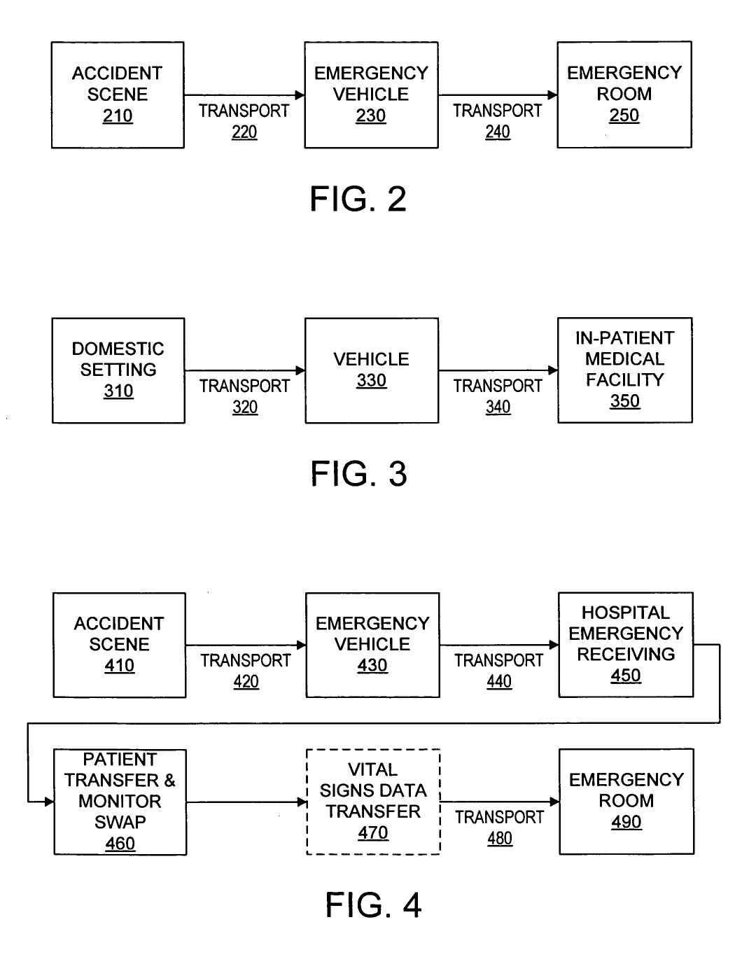

The vital signs monitor 110 is transportable so that the sensors 130, 140 and 150 may be attached to a patient at the beginning of an event and accompany the patient throughout the event. FIG. 2 shows an example in which the vital signs monitor 110 is attached to a gurney and the sensors 130, 140 and 150 are applied to a patient at an accident scene 210, for example, and remain with the patient during transport 220 to an emergency vehicle 230 such as an ambulance or helicopter, during the ride in the emergency vehicle 230, during transport 240 from the emergency vehicle 230 to a hospital emergency room 250, and even in the emergency room 250 if desired. The unit may even accompany the patient as the patient is transported within the hospital, as from department to department, as well as to the patient's room. When used in this manner, the vital signs monitor 110 records continuous or essentially continuous vital signs data, which may be displayed to the caregiver on demand, or which may be communicated to the patient's electronic record as maintained by the hospital, or generally communicated in any desired way for any desired use. Because this application is in a emergency environment which may involve a patient ranging from a child or frail elderly patient to a morbidly obese patient, preferably the sensors used in the vital signs monitor are universal.

FIG. 3 shows another application in which the monitor 110 is made smaller and lighter so as to be ambulatory. Consider a patient such as, for example, a morbidly obese patient requiring care at a domestic setting 310, such as the patient's house or apartment, a nursing home, or an assisted living residence. An ambulatory vital signs monitor may be carried by the patient at the domestic setting 310 and remains with the patient as the patient walks or is transported 320 to a vehicle 330, as the patient is transported by the vehicle 330, and as the patient walks or is transported 340 to a medical facility 350 such as a doctor's office, clinic or hospital. Advantageously, the physician or other caregiver at the medical facility 350 can inspect a history of the patient's vital signs directly on the ambulatory monitor, on a printout if the monitor is so equipped, or directly on the facility's computers if the facility is able to receive data transfer from the ambulatory monitor. Moreover, vital signs data may be transferred in real time or near real time to the facility's systems, so that the caregiver may monitor the patient's vital signs on a familiar system. Because this application may involve a morbidly obese patient, the use of a sensor-based wrist-mounted noninvasive blood pressure sensor such as the Vasotrac NIBP monitor is particularly advantageous.

In some cases, procedure calls for the ambulance's equipment to remain with the ambulance, and the hospital's equipment to remain with the hospital. FIG. 4 shows an application in which two transportable vital signs monitors are involved, the first as used by the ambulance crew, and the second as used by the hospital staff. The ambulance-owned vital signs monitor is attached to a gurney and the vital signs sensors are applied to a patient at an accident scene 410, for example, and remain with the patient during transport 420 to an emergency vehicle 430 such as an ambulance or helicopter, as well as during the ride in the emergency vehicle 430 and during transport 440 from the emergency vehicle 430 to a hospital emergency receiving area 450. At this time, the sensors of the ambulance-owned monitor are removed from the patient, the patient is transferred to a hospital gurney, and the sensors of a hospital-owned monitor are applied to the patient (block 460). Optionally, the patient's historic vital signs data is transferred from the ambulance-owned vital signs monitor to the hospital-owned vital signs monitor (block 470). The hospital-owned vital signs monitor is attached to the hospital gurney, and accompanies the patient during transport 480 from the hospital emergency receiving 450 into the emergency room 490.

To achieve transfer between monitors of a patient's historic vital signs data, the monitor 110 may be provided with a peer-to-peer communications capability, through which the monitor may transmit and receive control and data signals directly with another similarly-equipped vital signs monitor, quickly and seamlessly and without reliance on an external network. A low power wireless communication protocol such as Bluetooth is preferred, since such a protocol provides for units in proximity with one another to automatically seek and connect to one another without excessive power drain. However, other wireless protocols that support peer-to-peer may be used if desired, including such known protocols as 802.11a, 802.11b, 802.11g, Appletalk, Pre-N, and so forth.

While a wireless protocol is preferred because it avoids the risk of loss or damage to a cable, which would have to be kept with the vital signs monitor, a cable may be used if desired, along with any suitable wired communications protocol that supports direct transfer between devices. Illustratively, a cable connector 18 may be provided on the monitor 110. Alternatively, a pull-out cable may be used.

Optionally, the vital signs monitor 110 may be provided with wired or wireless networking capability, such networking systems being well known in the art.

The NIBP sensor unit 150 preferably is motion-tolerant. Suitable motion compensation techniques include the manual technique used in the Vasotrac® model AMP205A (Revision K) NIBP monitor, and the automatic technique used in the Vasotrac® model AMP205A (Revision L) NIBP monitor, which are available from Medwave Inc. of Danvers, Mass.; see, e.g., Medwave Inc., Vasotrac Model APM205A Non-Invasive Blood Pressure Monitor Operator's Manual, Revision K, May 2004; and Medwave Inc., Vasotrac Model APM205A Non-Invasive Blood Pressure Monitor Operator's Manual, Revision L, December 2004. The Vasotrac NIBP monitors incorporated a High Motion Tolerance (“HMT”) function that uses an adaptive noise canceling (“ANC”) algorithm on high-pass filtered signals from the main source and the ring source. The high pass filter generally corrects high, constant motion noise which may occur from activity at slow treadmill speeds. The ANC algorithm relies on the main-to-ring noise correlation to correct large noise levels which may occur from fast walking or running on a treadmill. The automatic technique is described in further detail in a U.S. patent application entitled “Noninvasive blood pressure monitor having automatic high motion tolerance,” which was filed May 2, 2005 and names Donna R. Lunak and Robert S. Bryngelson as inventors (Attorney Docket No. 01845.0047-US-01), and which hereby is incorporated herein in its entirety by reference thereto. Motion compensation techniques are also described in U.S. Pat. No. 6,132,382 entitled “Non-Invasive Blood Pressure Sensor with Motion Artifact Reduction” which issued Oct. 17, 2000 to Archibald et al., and U.S. Pat. No. 6,245,022 entitled “Non-Invasive Blood Pressure Sensor with Motion Artifact Reduction and Constant Gain Adjustment During Pressure Pulses” which issued Jun. 12, 2001 to Archibald et al., which hereby are incorporated herein in their entirety by reference thereto.

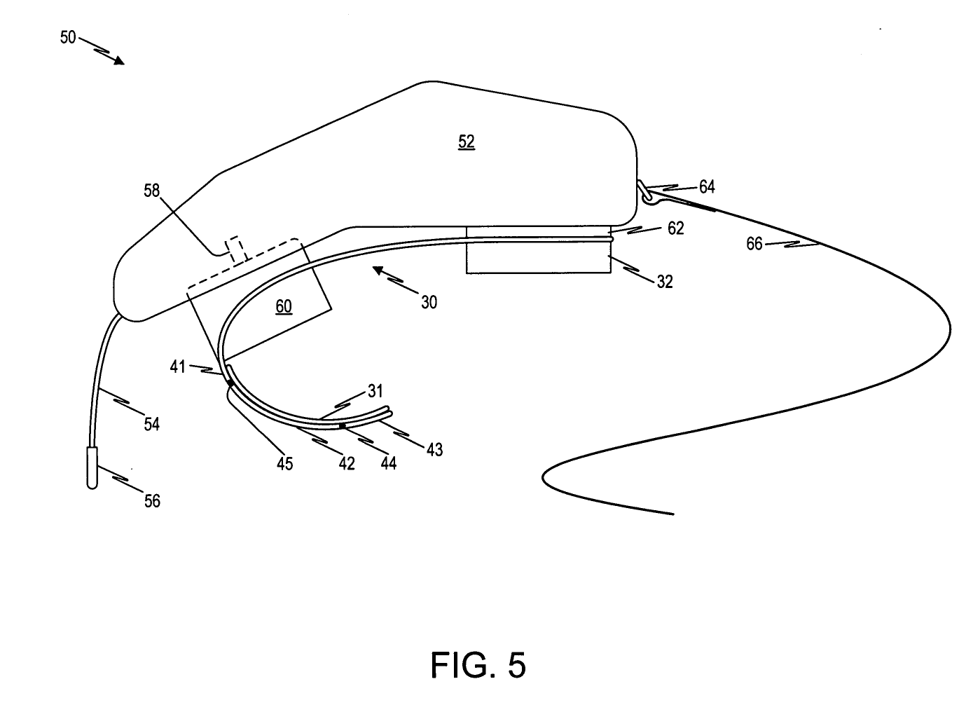

Advantageously, the vital signs monitor 110 is compatible with a broad range of patient sizes. This is made possible by the use of a noninvasive blood pressure sensor unit that may be applied to the patient at various extremities such as the wrist, the inside elbow, the ankle, and the top of the foot, the dimensions of which tend to vary less from patient-to-patient than other parts of the human anatomy. Where the noninvasive blood pressure sensor unit includes placement aids, such placement aids are also designed to work over a broad range of patient sizes. An illustrative universal pressure sensor unit with an articulated guide that accommodates wrist sizes in the range of about 11 centimeters to about 22 centimeters is shown in FIG. 5.

While various suitable types of blood oxygen saturation sensors are and will become available, a particularly suitable blood oxygen saturation sensor is the type that clips onto a patient's finger tip. While the sensors are typically available in three sizes—adult, pediatric, and neonatal—the range of patient sizes having wrist sizes in the range of about 11 centimeters to about 22 centimeters is adequately covered by the “adult” size, making the typical SpO₂ sensor “universal” as well. Suitable SpO2 sensor units include model MAX-A with adhesive D-25, model MAX-AL with adhesive D-25L, model MAX-R with adhesive R-15, model MAX-FAST, model OxiCliq A, model DS-100A, and model D-YSE, and suitable interface circuitry for the SpO2 sensor unit include model Oximax MP100, all of which are available from Nellcor Puritan Bennett Inc. of Pleasanton, Calif.

While various suitable types of temperature sensors are and will become available, a particularly suitable temperature sensor is the type that is placed in the patient's ear, in as much as the sensor is easy to use and the same-sized sensor works for both smaller and larger patients. This type of sensor is typically inserted into a patient's ear, and functions essentially independent of patient weight or size. A suitable model of temperature sensor is the Genius Model 8300G Tympanic Thermometer, which is available from Sherwood Davis & Geck of Watertown, N.Y.

Blood pressure may be determined from a sensor-based monitoring device that non-invasively senses at the surface of a patient's body pressure pulses that are influenced by blood flow in an underlying artery. A user positions the sensor on the wrist over the edge of the radius bone using an articulated placement guide. As varying hold-down pressure is applied so that the properly positioned sensor compresses tissue overlying the artery, pressure pulses are sensed by the sensor to produce data for various purposes, such as calculation of blood pressure and display of pulse waveform. Noninvasive sensor-based monitors for monitoring blood pressure, including systolic pressure, diastolic pressure, and pulse, are described in U.S. Pat. No. 5,797,850 issued Aug. 25, 1998 to Archibald et al., U.S. Pat. No. 5,640,964 issued Jun. 24, 1997 to Archibald et al., and U.S. Pat. No. 6,558,335 issued May 6, 2003, to Thede, which hereby are incorporated herein in their entirety by reference thereto. Placement guides may advantageously be used with these as well as other types of sensor-based monitors.

FIG. 5 is an edge view of a sensor unit 50 that has a housing 52 that contains a hold-down assembly (not shown) which includes a pair of generally parallel bale cords 54 and a bale 56. A sensor 60 is pivotally connected to the hold-down assembly by a pivot rod 58. An articulated placement guide 30 is used to properly position and stabilize the sensor 60 on the wrist of a patient. An illustrative articulated placement guide is described in further detail in a copending U.S. patent application Ser. No. 11/072,199 filed Mar. 4, 2005 (Kevin R. Evans, “Articulated placement guide for sensor-based noninvasive blood pressure monitor”) which hereby is incorporated herein in its entirety by reference thereto.

The sensor unit 50 is secured to the patient in any convenient manner, illustratively by strapping it on with a Velcro® brand strap 66. The ends of the strap 66 are looped through bale 56 and anchor 64, which are attached at or near opposite ends of the sensor unit 50. The anchor 64 is illustratively a U-shaped metal bracket that rotatably projects from the casing 52. The bale 56 is a slotted plastic body which is molded about the pair of bale cords 54, and receives the end of the strap 66.

To locate the proper position for placement of the sensor 60, the user first palpates the wrist with a finger to find the distal edge of the radius bone. The sensor 60 is then placed directly over this point, and the strap 66 is secured snugly. The articulated placement guide 30 that includes articulated segments 41, 42 and 43 helps in the proper placement. The placement guide is attached at one end of the 41 to the casing 52 by the mounting block 62. When the sensor unit 50 is applied to the patient, the placement guide 30 straddles the styloid process bone of the patient and generally guides the sensor 60 into position over the underlying artery and the radius bone. Indicator symbols such as notch symbols on the placement guide segment 42 (not shown) and an arrow symbol on the sensor 60 (not shown) align to the distal edge of the radius bone when the sensor 60 is properly positioned, and proper placement may be verified tactilely by passing a finger between the bail cords 54 and an access notch in the placement guide segments 41 and 42, and feeling the distal edge of the radius bone. The access notch extends from a generally circular aperture through which the sensor 60 moves. The portions of segment 42 that flank the aperture and access notch may be thought of as guide ribs which meet within segment 42.

When a monitoring cycle is initiated, a varying force is applied to the radial artery by the hold-down assembly, and the counter pressure in the radial artery produces a signal that is digitized and used to calculate blood pressure. Measurements may be made over one or more cycles, to perform spot monitoring or continuous monitoring. As the hold-down assembly operates, it draws in the bale 56 via the bale cords 54, so that sensor 60 gently exerts pressure against the patient's wrist over the radial artery, while cushion 32 on the placement guide segment 41 and layer 31 extending across whole or parts of placement guide segments 41, 42 and 43 and spanning intervening gaps 44 and 45 gently distribute pressure over other areas of the patient's wrist. The cushion 32 also functions as a pivot point about which the hold-down pressure is applied, while the layer 31 also enables articulation.

Since the sensor 60 is relatively small compared to such devices as cuffs used with the oscillometric and auscultatory methods, the sensor 60 applies a hold down pressure to only a relatively small area above the underlying artery of the patient. Consequently, blood pressure measurements may be taken with less discomfort to the patient, and downstream measurements such as blood oxygen saturation taken with a finger clip are not affected. Because the sensor 60 does not require inflation or deflation, faster and more frequent measurements may be taken. Furthermore, the sensor 60 better conforms to the anatomy of the patient so as to be more comfortable to the patient, and the improved accuracy and repeatability of placement and the automatic application of the hold-down pressure avoids ineffective hold-down cycles and achieves consistent and accurate blood pressure measurements.

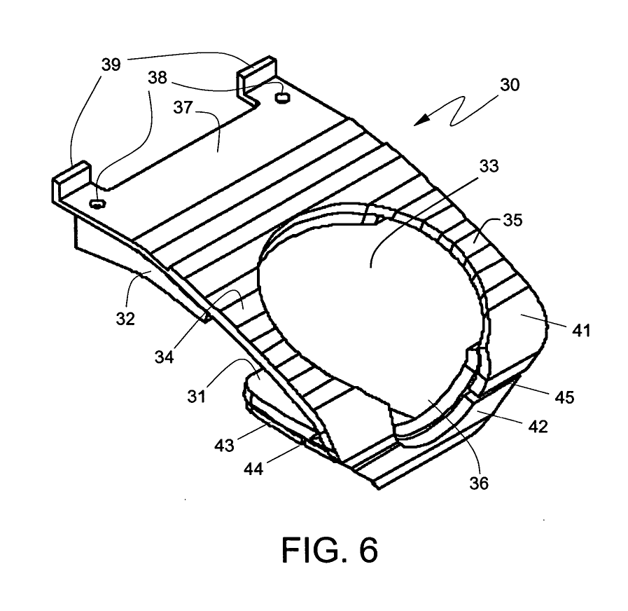

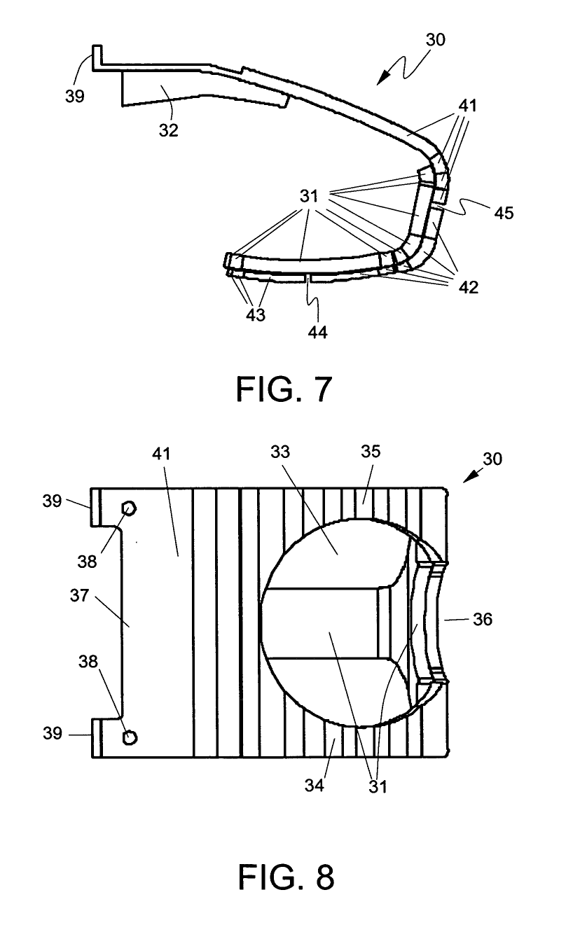

FIGS. 6-8 show three different views of a “universal” articulated placement guide 30. The articulated placement guide 30 is divided into three segments 41, 42 and 43, at articulation regions 44 and 45. Because of the articulation regions 44 and 45, the range over which the articulated placement guide 30 can expand to fit the wrist snugly is greater than earlier placement guides, thereby eliminating the need for several different-sized placement guides to cover a full range of the most common patient sizes. In this manner, a single articulated placement guide 30 can accommodate a variety of different patients, resulting in greater convenience and less expense for the sensor unit 50.

Although three segments are shown in the articulated placement guide 30, it will be appreciated that any suitable number of segments may be used, including two, four, and greater than four.

While the articulation regions 44 and 45 may be formed in any of a variety of different ways, one simple and effective technique is to use a continuous flexible inner layer 31 that extends across all or parts of the three segments 41, 42 and 43. Preferably, the inner layer 31 is a single piece of shaped material that tends to return to its original shape after being flexed, which increases comfort for the patient and strength of the articulated placement guide 30. Alternatively, separate pieces in a discontinuous layout may be used to join adjacent segments. A preferable material for the inner layer 31 is polyurethane with polyester threads embedded in it, which enhances strength, flexibility and durability.

While the articulated placement guide 30 may be made in any of a variety of different ways, one technique is to form the inner layer 31 as a curved polyurethane strip with embedded polyester threads, and then place the strip in a mold for overmolding the inner layer 31 with segments of any suitable plastic such as Kydex® thermoplastic, which is a thermoplastic alloy. Although the plastic segments made with Kydex thermoplastic have some flex, they are generally rigid and have much less flexibility than the polyurethane strip, which makes the articulated placement guide 30 easier to work with during application of the monitoring 50 device to the patient. However, if desired an even more rigid material may be used for the segments.

An articulated placement guide such as the guide 30 having 3 segments and two articulation regions may be dimensioned as follows to fit a range of wrist circumferences from about 11 cm to about 22 cm: length of about 13 cm, minimum width of about 1.3 cm, maximum width of about 4.8 cm, minimum segment thickness about 1.27 mm (50 mils), maximum segment thickness (generally about the aperture 33) of about 2.29 mm (90 mils), and thickness of the inner layer 31 of about 2.54 mm (100 mils). The range of wrist sizes from about 11 cm to about 22 cm is sufficient to include a broad spectrum of patients, ranging from children weighing as little as 43 pounds to obese and bariatric patients. This range covers about 95% of all patients.

The cushion 32 is affixed to the inside of segment 41 to provide comfort for the patient and to stabilize the sensor unit 50 on the patient's wrist. The cushion 32 may be made from any suitable material.

The articulated placement guide 30 further has a sensor aperture 33 which allows the sensor 60 (FIG. 5) to pass through the articulated placement guide 30 and contact the patient. Guide ribs 34 and 35 are provided on the sides of the sensor aperture 33. A notch 36 extends from the aperture 33 so that during placement of the sensor unit 50 on the wrist, the patient or practitioner adjusts the device until he or she can feel the distal end of the radius bone with a finger placed through the notch 36 to ensure proper placement. Segment 41 has a ledge 37 for engagement with other mechanical parts of the device, as well as screw holes 38 and ridges 39, for attachment to the sensor unit 50. It will be appreciated that other methods for attachment may be used in addition to, or instead of elements 37, 38 and 39. The attachment may be removable or permanent.

The articulated placement guide 30 may be made from a variety of suitable materials, including plastics, rubbers, and metals. In one variation, a thin springy stainless steel may be used for the inner layer 31, with a suitable segmented overcoat of a protective and cushioning material. In other variations, the articulated placement guide hinges may be realized by a continuous sheet of flexible material that has a predetermined shape and a tendency to return to that shape when flexed, but which is thinned or otherwise made more flexible in limited predetermined places to form the articulation regions.

It will be appreciated that the sensor in the sensor unit may be unitary, or various components of the sensor may be distributed elsewhere in the sensor unit. Where the sensor includes a pressure transducer, for example, the pressure transducer may be mounted to a supporting member of the sensor that also supports the pressure transmission medium containing the sensing surface, or may be mounted to a supporting member elsewhere in the device and placed in fluid communication with the sensing surface through a fluid-filled tube.

It will be appreciated that although the articulated placement guide is described herein in the context of a wrist-mounted monitoring device, the monitoring device and the associated articulated placement guide may be designed for use with other anatomical structures on which noninvasive monitoring for blood pressure may be performed over a broad range of patient sizes, including children, the elderly, adults, and morbidly obese patients. Such anatomical structures include the inside elbow, the ankle, and the top of the foot.

The description of the invention and its applications as set forth herein is illustrative and is not intended to limit the scope of the invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. An apparatus for monitoring vital signs of pediatric, adult, and morbidly obese patients, comprising:
a transportable control unit;
a universal noninvasive patient blood oxygen saturation sensor unit coupled to the control unit;
a universal noninvasive patient temperature sensor unit coupled to the housing; and
a universal patient noninvasive blood pressure sensor unit coupled to the housing.

2. The apparatus of claim 1 wherein:
the blood pressure sensor unit is a wrist-mounted type that is usable without modification over a range of pediatric, adult and morbidly obese patients having a wrist size in a range of about 11 cm to about 22 cm;
the patient blood oxygen saturation sensor unit is usable without modification over the range of pediatric, adult and morbidly obese patients; and
the patient temperature sensor is usable without modification over the range of pediatric, adult and morbidly obese patients.

3. The apparatus of claim 1 wherein the patient blood oxygen saturation sensor, the patient temperature sensor, and the patient noninvasive blood pressure sensor are wirelessly coupled to the control unit.

4. The apparatus of claim 1 wherein the patient blood oxygen saturation sensor, the patient temperature sensor, and the patient noninvasive blood pressure sensor are coupled to the control unit by respective cables.

5. The apparatus of claim 1 wherein the control unit comprises a short range wireless transceiver for peer-to-peer transfer of patient data, including patient blood pressure, oxygen saturation, and temperature.

6. The apparatus of claim 1 wherein:
the blood oxygen saturation sensor is a clip type for a fingertip;
the temperature sensor is a tympanic type for an ear; and
the blood pressure sensor is a wrist type comprising:
a body;
a hold-down assembly incorporated into the body;
a sensor element pivotally extending from the body; and
a articulated placement guide extending from the body for guiding placement of the sensor element upon the distal edge of the radius bone, the articulated placement guide being suitable for wrist sizes over a range of about 11 cm to about 22 cm.

7. The apparatus of claim 1 wherein the control unit further comprises processing circuitry for generating motion-compensated pressure waveform trend data from measurements by the blood pressure sensor unit, and for calculating motion-compensated systolic pressure, diastolic pressure, and mean pressure values from measurements by the blood pressure sensor unit.

8. An apparatus for monitoring vital signs of pediatric, adult, and morbidly obese patients, comprising:
a transportable control unit having a display;
a noninvasive patient blood pressure sensor unit usable without modification over a range of pediatric, adult and morbidly obese patients, the control unit being suitable for wrist sizes over a range of about 11 cm to about 22 cm and coupled to the control unit;
a patient blood oxygen saturation sensor unit usable without modification over the range of pediatric, adult and morbidly obese patients and coupled to the control unit; and
a patient temperature sensor unit usable without modification over the range of pediatric, adult and morbidly obese patients and coupled to the control unit;
wherein the control unit comprises processing circuitry for generating motion-compensated pressure waveform trend data from measurements by the blood pressure sensor unit, for calculating motion-compensated systolic pressure, diastolic pressure, and mean pressure values from measurements by the blood pressure sensor unit, for generating pulse oximetry waveform data from measurements by the blood oxygen saturation sensor unit, and for calculating oxygen saturation values from measurements by the blood oxygen saturation unit; and
wherein the control unit comprises display circuitry for displaying the pressure waveform trend data, the pulse oximetry waveform data, and the oxygen saturation, systolic pressure, diastolic pressure, and mean pressure values on the display.

9. The apparatus of claim 8 wherein the patient blood oxygen saturation sensor, the patient temperature sensor, and the patient noninvasive blood pressure sensor are wirelessly coupled to the control unit.

10. The apparatus of claim 8 wherein the patient blood oxygen saturation sensor, the patient temperature sensor, and the patient noninvasive blood pressure sensor are coupled to the control unit by respective cables.

11. The apparatus of claim 8 wherein the control unit comprises a short range wireless transceiver for peer-to-peer transfer of patient data, including patient blood pressure, oxygen saturation, and temperature.

12. An apparatus for monitoring vital signs of a patient, comprising:
a transportable housing;
a clip-type fingertip patient blood oxygen saturation sensor mechanically coupled to the housing;
a tympanic patient temperature sensor mechanically coupled to the housing;
a noninvasive wrist-mounted blood pressure sensor unit mechanically coupled to the housing, the blood pressure sensor comprising an articulated placement guide suitable for wrist sizes over a range of about 11 cm to about 22 cm;
a display mounted in the housing, the display having an LCD portion for displaying information graphically, and an LED portion for displaying information alphanumerically;
processing circuitry contained in the housing and electrically coupled to the blood oxygen saturation sensor, the temperature sensor, and the noninvasive blood pressure sensor for determining patient blood oxygen saturation, patient temperature, and patient blood pressure;
display circuitry contained in the housing and electrically coupled to the processing circuitry for displaying waveforms indicative of the patient blood oxygen saturation and the patient blood pressure on the LCD portion of the display, and for displaying alphanumeric values indicative of the patient blood oxygen saturation, patient temperature, and the patient blood pressure on the LED portion of the display; and
communications circuitry contained in the housing and electrically coupled to the processing circuitry for transmitting the patient blood oxygen saturation, the patient temperature, and the patient blood pressure using a wireless protocol.

13. The apparatus of claim 12 wherein the communications circuitry is compliant with a Bluetooth protocol.

14. The apparatus of claim 13 wherein the communications circuitry is further compliant with an Ethernet protocol.

15. A method of monitoring a patient for vital signs, comprising:
acquiring vital signs of a patient during transport in a motorized vehicle with a transportable vital signs monitor system having universal vital sign sensors; and
continuing to acquire the vital signs of the patient with the transportable vital signs monitor system and the universal vital sign sensors in an emergency room.

16. The method of claim 15 wherein the motorized vehicle is an ambulance.

17. The method of claim 15 wherein the motorized vehicle is an aircraft.

18. A method of monitoring a patient for vital signs, comprising:
acquiring a first set of vital signs of a patient in a motorized vehicle with a first transportable vital signs monitor system having universal vital sign sensors;
acquiring a second set of vital signs of the patient with a second transportable vital signs monitor system having universal vital sign sensors in a stationary examination location; and
transferring the first set of vital signs from the first transportable vital signs monitor to the second transportable vital signs monitor while the patient is being transferred from the motorized vehicle to the stationary examination location.

19. The method of claim 18 wherein the motorized vehicle is an ambulance, and wherein the stationary examination location is an emergency room.

20. The method of claim 18 further comprising:
acquiring a third set of vital signs of the patient in a domestic setting with the first transportable vital signs monitor system, prior to acquiring the first set of vital signs; and
transferring the third set of vital signs from the first transportable vital signs monitor to the second transportable vital signs monitor while the patient is being transferred from the motorized vehicle to the stationary examination location.
